(19) United States
(12) Patent Application Publication
(10) Pub. No.: US 2011/0137272 A1
(43) Pub. Date: Jun. 9, 2011

(54) Safety Ring Lid Closure

(75) Inventor: Chuck Adams, Naperville, IL (US)

(73) Assignee: Medical Action Industries, Incorporated, Brentwood, NY (US)

(21) Appl. No.: 12/630,211

(22) Filed: Dec. 3, 2009

(51) Int. Cl. A61F 5/44; B65D 43/16

(52) U.S. Cl. 604/349; 220/810

(57) ABSTRACT

A closure for a blow-molded container is disclosed, comprising a ring portion, a hinge portion, and a lid portion. Also disclosed are combinations of a closure and a blow-molded container, which in certain embodiments is a blow-molded urinal.

### CROSS-REFERENCE TO RELATED APPLICATION

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

2. Description of the Related Art

One method of manufacturing one piece, all plastic containers is by using an extrusion blow molding process. In this process, a molten tube of plastic is dropped between two halves of a mold. The mold halves are closed onto the molten tube. Then, compressed air is injected into the molten tube forcing the walls of the tube to expand into the mold, thus taking the shape of the cavity in the mold. The mold and molten plastic are cooled so that the plastic retains the permanent shape of the mold.

When the mold halves are open, the plastic container is removed from the mold. A flashing of excess plastic is trimmed from the parting line where the two halves of the mold come together, including the parting line at the neck or opening end of the container. It is most difficult to trim consistently especially in the neck area because the flashing material is the thickest at this point. The trim line at the neck or opening of the container can be sharp, which can cause a cut or abrasion to the skin when any human appendage (or penis in the case of a urinal) is inserted into the container. There can also be possible injury to the skin on other parts of the body coming into contact with a sharp rim or excess flash. However, since blow molding is in many instances the only practical way of making a one piece plastic container at a cost effective price, an elimination of the occasionally sharp edge at the neck or rim of the container is needed. Therefore, a method of eliminating or covering the potentially sharp edge at the neck, rim, or opening of the container is highly desired.

### BRIEF SUMMARY OF THE INVENTION

The present disclosure overcomes the risk of injury associated with the potentially sharp or rough edges of blow-molded containers by providing closures that cover or surround such sharp or rough edges.

The present disclosure provides a closure, comprising a ring comprising a side wall comprising an outer surface, an inner surface, and a bottom surface, a top wall comprising a first end, a second end, an upper surface, and a lower surface, the side wall connected to the first end of the top wall, and a lip comprising an inner surface, an outer surface, and a tip, the lip connected to the second end of the top wall and defining an opening, a hinge comprising a first end and a second end, the first end of the hinge connected to the outer surface of the side wall of the ring, and a lid comprising a base comprising an outer edge, and a raised portion comprising an upper end, a lower end, and an outer wall, the lower end of the raised portion inset from the outer edge of the base and connected to the base, the raised portion adapted to releasably engage the lip of the ring, the outer edge of the base of the lid connected to the second end of the hinge.

In certain aspects of the disclosure, the lip is generally parallel to the side wall. In other aspects, the inner surface of the side wall comprises a projection, which in various embodiments can be located at a position lower (further from the top wall of the ring) than the tip of the lip, about equivalent to (about the same distance from the top wall of the ring) the tip of the lip, or higher (closer to the top wall of the ring) than the tip of the lip. In particular aspects of the disclosure, the opening defined by the lip of the ring is generally circular in shape, although the opening can take any shape in other embodiments, including, but not limited to, a circle with a flattened side, an oval, an oval with a flattened side, a polygon, such as a triangle, a square, a rectangle, a pentagon, or a hexagon. In further embodiments, the outer surface of the side wall of the ring further comprises a bump-out.

In some embodiments, the hinge is flexible, and can have a length less than the length of the ring portion, about equal to the ring portion, or longer than the ring portion of the closure. In certain aspects of these embodiments, the first end of the flexible hinge is attached to the outer surface of the side wall of the ring adjacent the bottom surface of the side wall of the ring, while in other aspects the first end of the flexible hinge is attached to the outer surface of the side wall of the ring adjacent the top surface of the side wall of the ring. In yet other aspects, the hinge is a generally pliable living hinge. In other embodiments, the hinge is stiff, and comprises an indent, which can be located at any position along the length of the hinge, including near the ring portion, about a quarter of the distance between the ring and the lid, about halfway between the ring and the lid, near the middle of the hinge, about 75% of the distance between the ring and the lid, or near the lid portion of the closure.

In particular aspects of the disclosure, the lid further comprises a flap comprising an upper surface and a lower surface, the flap attached to the outer edge of the base of the lid. In certain embodiments, the second edge of the hinge and the flap attach to different regions of the outer edge of the base portion of the lid. For example, the second edge of the hinge and the flap are attached to the outer edge of the base portion of the lid opposite each other. In further embodiments, the flap comprises at least a first ridge. In some aspects, the upper surface of the flap comprises at least a first ridge or a plurality of ridges, in other aspects the lower surface of the flap comprises at least a first ridge or a plurality of ridges, while in still other aspects the upper surface and the lower surface of the flap each comprise at least a first ridge or a plurality of ridges.

In certain embodiments of the disclosure, the outer wall of the raised portion of the lid comprises at least a first protuberance or a plurality of protuberances. In particular embodiments, the at least a first protuberance is, or plurality of protuberances are, located proximate the upper end of the raised portion of the lid.

In various embodiments of the disclosure, the closure is plastic, wood, metal, rubber, latex, or a paper-based product. In certain embodiments, the closure is plastic, including, but not limited to, polyethylene, polypropylene, an engineering grade plastic, including, but not limited to, engineering grade acrylonitrile-butadiene-styrene, polyurethane, nylon, polycarbonate, polyethylene, polyphenylsulfone, polyoxymethylene, acetal copolymer, polysulfone, polycarbonate/acrylonitrile-butadiene-styrene, polyetherimide, polybutylene terephthalate, polyphenylene/polystyrene, polyphenylene sulfide, polycarbonate/polybutylene terephthalate, polycarbonate/polyethylene terephthalate, or an engineering grade thermoplastic elastomer, a bioplastic, including, but not limited to, a starch-based plastic, a polylactic acid plastic, poly-3-hydroxybutyrate plastic, polyamide 11, a bio-derived polyethylene plastic, or a genetically modified bioplastic, or any combination thereof. In some aspects, one or two of the components (ring, hinge, lid) of the closure can be manufactured from a different material than the other component or components. In further aspects, each of the components of the closure is manufactured from different materials.

In certain embodiments of the disclosure, the closure is comprised within an adapter for placement into the opening of a blow molded container.

The present disclosure also provides a blow molded container comprising a body, a rim connected to the body and defining an opening, and a closure, the closure comprising a ring comprising a side wall comprising an outer surface, an inner surface, and a bottom surface, a top wall comprising a first end, a second end, an upper surface, and a lower surface, the side wall connected to the first end of the top wall, and a lip comprising an inner surface, an outer surface, and a tip, the lip connected to the second end of the top wall and defining an opening, the ring engaged with the rim of the container, a hinge comprising a first end and a second end, the first end of the hinge connected to the outer surface of the side wall of the ring, and a lid comprising a base comprising an outer edge, and a raised portion comprising an upper end, a lower end, and an outer wall, the lower end of the raised portion inset from the outer edge of the base and connected to the base, the raised portion adapted to releasably engage the lip of the ring, the outer edge of the base of the lid connected to the second end of the hinge.

In certain embodiments of the disclosure, the ring is releasably engaged with the rim, while in other embodiments the inner surface of the side wall of the ring further comprises a projection, and the projection engages the rim of the container. In still other embodiments, the ring is attached to the rim using ultrasonic welding, spin welding, manual or mechanical securement, or an adhesive, including, but not limited to, solvent-type binding glues or adhesives, acrylic cement, and hot melt moisture curing adhesives, or any combination of attachment means or adhesives.

The disclosure additionally provides a blow molded container comprising a body, a neck connected to the body, a rim connected to the neck and defining an opening, and a closure, the closure comprising a ring comprising a side wall comprising an outer surface, an inner surface, and a bottom surface, a top wall comprising a first end, a second end, an upper surface, and a lower surface, the side wall connected to the first end of the top wall, and a lip comprising an inner surface, an outer surface, and a tip, the lip connected to the second end of the top wall and defining an opening, the ring engaged with the rim of the container, a hinge comprising a first end and a second end, the first end of the hinge connected to the outer surface of the side wall of the ring, and a lid comprising a base comprising an outer edge, and a raised portion comprising an upper end, a lower end, and an outer wall, the lower end of the raised portion inset from the outer edge of the base and connected to the base, the raised portion adapted to releasably engage the lip of the ring, the outer edge of the base of the lid connected to the second end of the hinge. In certain embodiments, the blow molded container is a urinal, for example a male or female urinal.

### BRIEF DESCRIPTION OF THE SEVERAL VIEWS OF THE DRAWINGS

For more complete understanding of the features and advantages of the present invention, reference is now made to the detailed description of the invention along with the accompanying figures.

FIG. 1. View from top of ring and bottom of lid of one embodiment of the disclosed closure.

FIG. 2. Side view of the embodiment of the disclosed closure shown in FIG. 1 through line A-A.

FIG. 3. View from bottom of ring and top of lid of the embodiment of the disclosed closure shown in FIG. 1.

FIG. 4. Edge view of the lid from the embodiment of the disclosed closure shown in FIG. 1 through line B-B, excluding the background hinge and ring portions of the closure for sake of clarity.

FIG. 5. Detail C showing side view of the ring from the embodiment of the disclosed closure shown in FIG. 2.

FIG. 6. Detail D showing side view of the flap from the embodiment of the disclosed closure shown in FIG. 2.

FIG. 7. View from top of ring and bottom of lid of second embodiment of the disclosed closure.

FIG. 8. Side view of the embodiment of the disclosed closure shown in FIG. 7 through line A-A.

FIG. 9. View from bottom of ring and top of lid of the embodiment of the disclosed closure shown in FIG. 7.

FIG. 10. Side view of third embodiment of the disclosed closure with indent in hinge.

FIG. 11. Side view of fourth embodiment of the disclosed closure with living hinge.

FIG. 12. Front view of one embodiment of the disclosed closure and blow-molded urinal, with the closure in the open position.

FIG. 13. Side view of the embodiment of the disclosed closure and blow-molded urinal shown in FIG. 12, with the closure in the open position, through line A-A.

FIG. 14. Detail B showing side view of the ring and urinal opening from the embodiment of the disclosed closure and blow-molded urinal shown in FIG. 13.

FIG. 15. Detail C showing side view of the ring, hinge, and urinal opening from the embodiment of the disclosed closure and blow-molded urinal shown in FIG. 13.

FIG. 16. Front view of one embodiment of the disclosed closure and blow-molded urinal, with the closure in the shut or closed position.

FIG. 17. Side view of the embodiment of the disclosed closure and blow-molded urinal shown in FIG. 16, with the closure in the shut or closed position, through line A-A.

FIG. 18. Detail B showing side view of the ring, flap, and urinal opening from the embodiment of the disclosed closure and blow-molded urinal shown in FIG. 17.

FIG. 19. Detail C showing side view of the ring, hinge, and urinal opening from the embodiment of the disclosed closure and blow-molded urinal shown in FIG. 17.

### DETAILED DESCRIPTION OF THE INVENTION

The present disclosure provides closures that cover or surround the sharp or rough edges of blow-molded containers, thereby overcoming some or all of the shortcomings present in current blow-molded containers, including, but not limited to, the risk of injury (laceration, abrasion, etc.) associated with the potentially sharp or rough edges. While the specific examples given here, for convenience of description and not limitation, is a blow-molded plastic urinal, it should be understood that the same principles apply to almost any blow-molded container having a less than smooth opening surface to be covered by the ring portion of the disclosed closure. Accordingly, the invention is applicable to and the claims are to be construed to cover all equivalent structures.

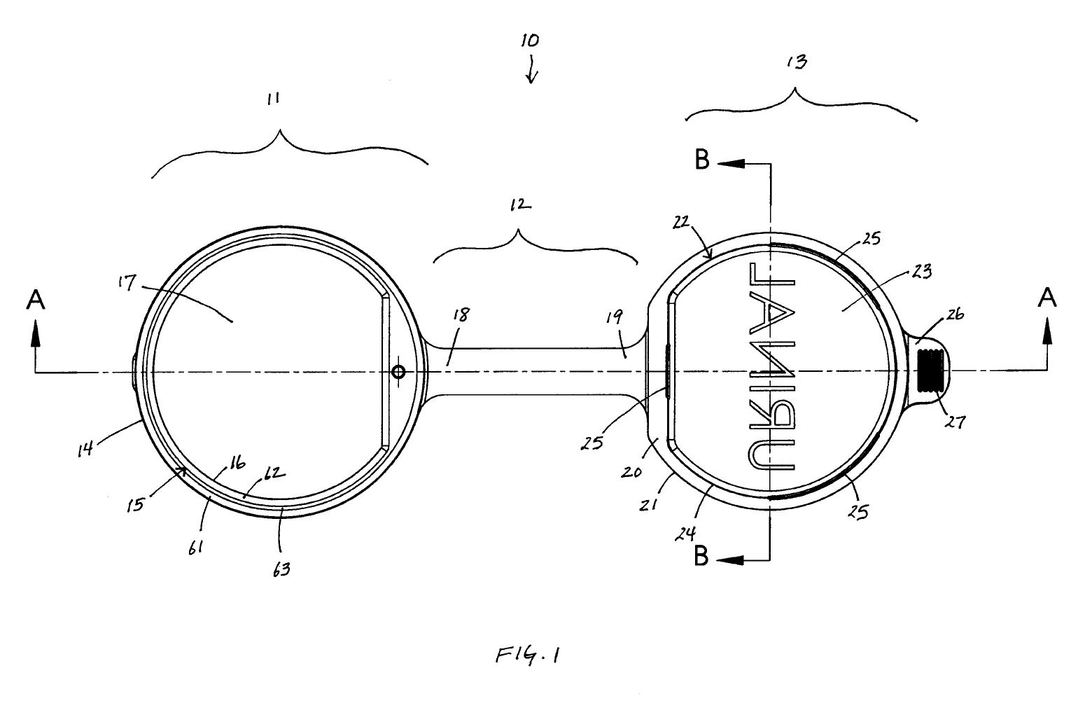

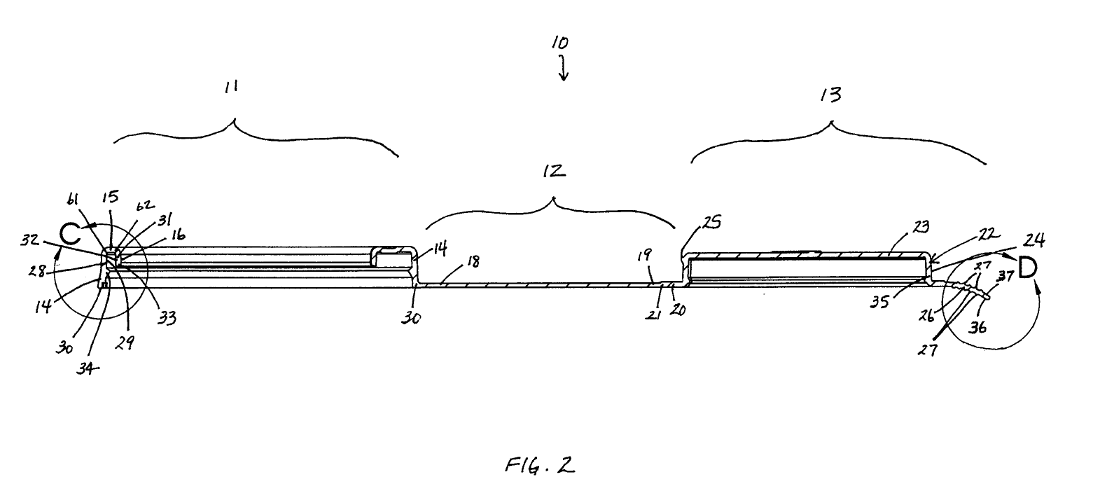

Turning to the figures, FIG. 1 shows a top view of one embodiment of the closure 10. The closure generally comprises three sections, the ring portion (or “ring”) 11, the hinge portion (or “hinge”) 12, and the lid portion (or “lid”) 13. As used in FIG. 1, the “top view” of the closure 10 is a view from the top of the ring 11 and the bottom of the lid 13. Ring 11 comprises a side wall (not visible in this view) comprising an outer surface 14, an inner surface (not visible in this view), and bottom surface (not visible in this view), a top wall 15, comprising a first end 61, a second end 62, an upper surface 63 and a lower surface (not visible in this view), and a lip (not visible in this view) having an outer surface 16 and an inner surface (not visible in this view). The outer surface of the lip 16 defines an opening 17. Lid 13 comprises a base 20 having an outer edge 21, a raised central portion 22 comprising an upper end 23 having an outer wall 24 having optional protuberances 25, and a flap 26 having a series of optional ridges 27. The flap 26 is connected to the outer edge 21 of the base 20 of the lid 13. Opening 17 of ring 11 is adapted to receive the raised central portion 22 of lid 13. Although in FIG. 1 the opening 17 is shown as a circle flattened on one end, the opening (and corresponding raised central portion of lid) can take any shape, including, but not limited to, circular, oval, oval flattened on one end, triangular, rectangular, square, pentagonal, hexagonal, heptagonal, octagonal, polygonal, or any other desired shape. The hinge portion 12 comprises a first end 18 connected to the outer surface 14 of the side wall of the ring 11 and a second end 19 connected to the outer edge 21 of the base 20 of the lid 13. In this embodiment of the closure 10, the first end of the hinge 18 is connected to the outer surface of the side wall 14 near the bottom surface of the side wall (not visible in this view). The connection of the hinge 12 to the ring 11 and the lid 13 is more clearly shown in FIG. 2. In this embodiment of the closure 10, the second end 19 of the hinge 12 is connected to the outer edge 21 of the base 20 of the lid 13 opposite (180° opposed to) the flap 26. However, in other embodiments of the closure 10 the second end 19 of the hinge 12 can be connected to the outer edge 21 of the base 20 of the lid 13 at a variety of different positions relative to the flap 26, including, but not limited to, about 15° opposed to, about 20° opposed to, about 25° opposed to, about 30° opposed to, about 35° opposed to, about 40° opposed to, about 45° opposed to, about 50° opposed to, about 55° opposed to, about 60° opposed to, about 65° opposed to, about 70° opposed to, about 75° opposed to, about 80° opposed to, about 85° opposed to, about 90° opposed to, about 95° opposed to, about 100° opposed to, about 105° opposed to, about 110° opposed to, about 115° opposed to, about 120° opposed to, about 125° opposed to, about 130° opposed to, about 135° opposed to, about 140° opposed to, about 145° opposed to, about 150° opposed to, about 155° opposed to, about 160° opposed to, about 165° opposed to, about 170° opposed to, or about 175° opposed to, or so.

FIG. 2 shows a side view of the embodiment of the closure 10 shown in FIG. 1 along line A-A. Once again closure 10 generally comprises three sections, ring 11, hinge 12, and lid 13. In this view, the side wall 28 of the ring 11 is shown, along with the outer surface 14, the inner surface 29 having an optional projection 34, and the bottom surface 30 of the side wall 28. Additionally, the top wall 15 of the ring comprising first end 61 and second end 62 is shown. Also shown is lip 31 having an outer surface 16, an inner surface 32 and a tip 33. The first end 18 of the hinge 12 is connected to the outer surface 14 of the side wall 28 near the bottom surface 30 of the ring 11, and the second end 19 of the hinge 12 is attached to the outer edge 21 of the base 20 of the lid 13. In this view, the raised portion 22 of the lid 13 is more clearly shown, with the upper end 23, a lower portion 35, and outer wall 24. Additionally, optional protuberance 25 on the outer wall 24 near the upper end 23 of the raised portion 22 of the lid 13 is shown. Also shown is flap 26 having an upper surface 36 and a lower surface 37, each of which include optional ridges 27. The number of the optional ridges 27 can vary from about 2 to about 10, including, but not limited to, 2, 3, 4, 5, 6, 7, 8, 9, 10, or more ridges. In other embodiments, the optional ridges 27 can be limited to only the upper surface 36 of the flap 26 or only the lower surface 37 of the flap 26. Additionally, although in this embodiments there are equal numbers of ridges 27 on the upper surface 36 and the lower surface 37 of the flap 26, in other embodiments there can be different numbers of ridges 27 on the upper surface 36 of and the lower surface 37 of the flap 26.

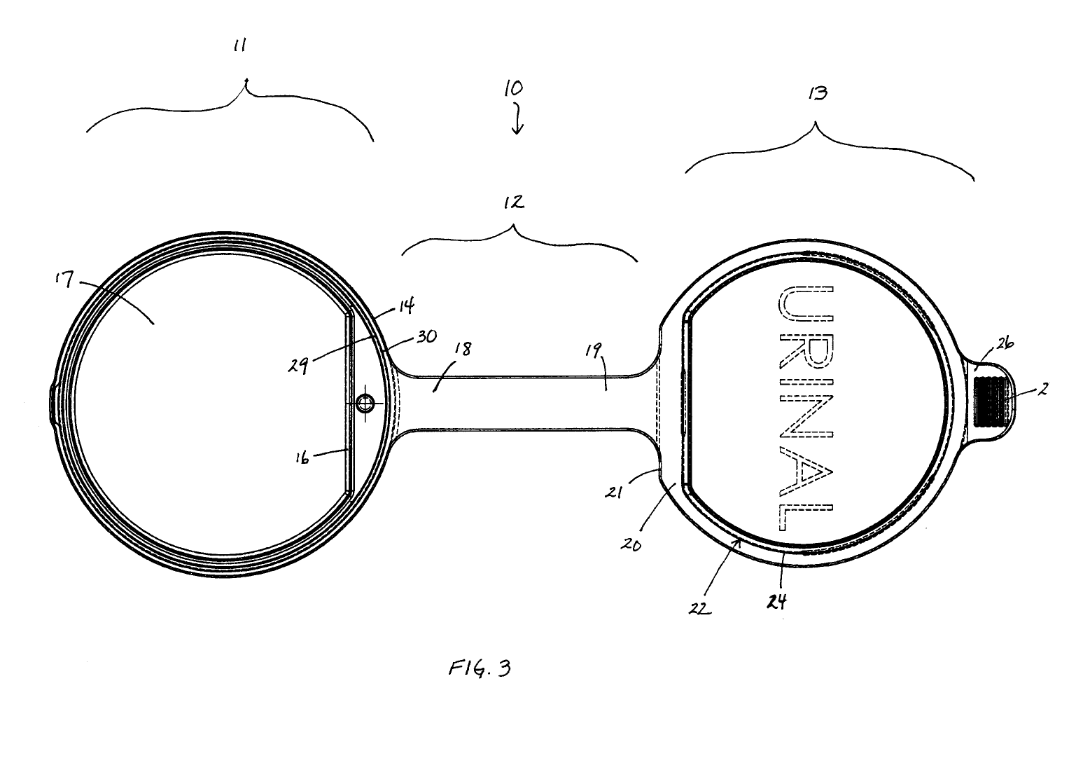

FIG. 3 shows a bottom view of the embodiment of the closure 10 shown in FIG. 1. Once again closure 10 generally comprises three sections, the ring 11, the hinge 12, and the lid 13. As used in FIG. 3, the “bottom view” of the closure 10 is a view from the bottom of the ring 11 and the top of the lid 13. Ring 11 comprises outer surface 14, inner surface 29, top surface (not visible in this view), and bottom surface 30 of the side wall (not visible in this view), top wall (not visible in this view), and lip (not visible in this view) having an outer surface 16 and tip (not visible in this view). The outer surface of the lip 16 defines an opening 17 that is adapted to receive the raised central portion 22 of the lid 13. The hinge portion 12 has a first end 18 connected to the outer surface 14 of the side wall near the bottom surface 30, and a second end 19 connected to the lid 13. The lid 13 has a base 20 having an outer edge 21 and a raised central portion 22 comprising an upper end (not visible in this view), a lower end (not visible in this view), an outer wall 24, and a flap 26 with a series of optional ridges 27. The second end 19 of the hinge portion 12 is connected to the outer edge 21 of the base 20 of the lid 13.

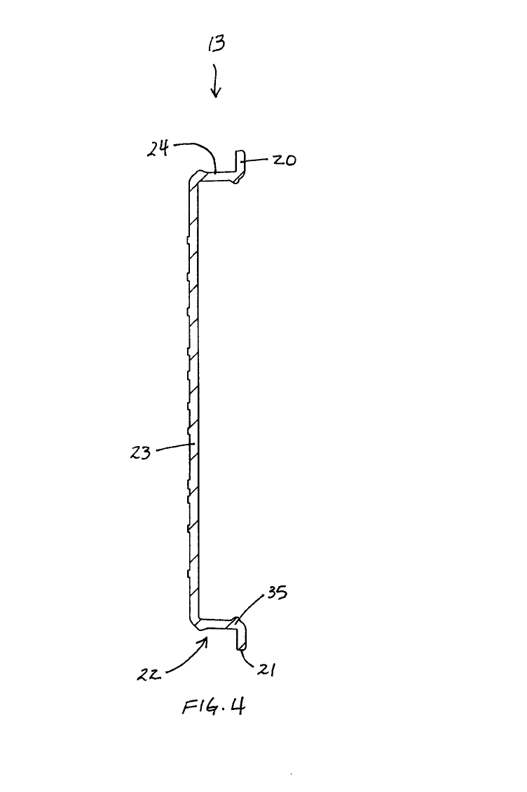

FIG. 4 shows an edge view of the lid 13 of one embodiment of closure 10, through line B-B of FIG. 1. In this view, which omits the background hinge and ring portions of the closure for sake of clarity, the base 20 and raised portion 22 of lid 13 are visible, as are the outer edge 21 of the base portion 20, the upper end 23, lower end 35 and outer wall 24 of raised portion 22.

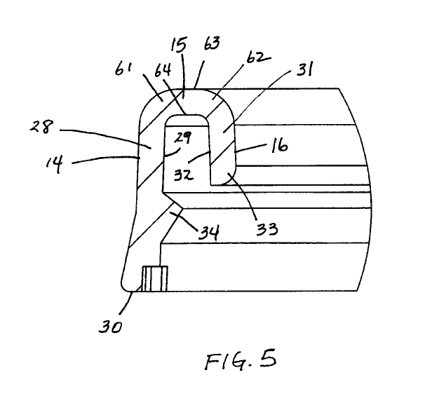

FIG. 5 shows an expanded view of detail C from FIG. 2, showing side view of the ring 11 from one embodiment of closure 10. More clearly visible in this view of ring 11 are side wall 28 comprising outer surface 14, inner surface 29, with optional projection 34, and bottom surface 30, top wall 15 comprising first end 61, second end 62, upper surface 63, and lower surface 64, and lip 31 with outer surface 16, inner surface 32, and tip 33.

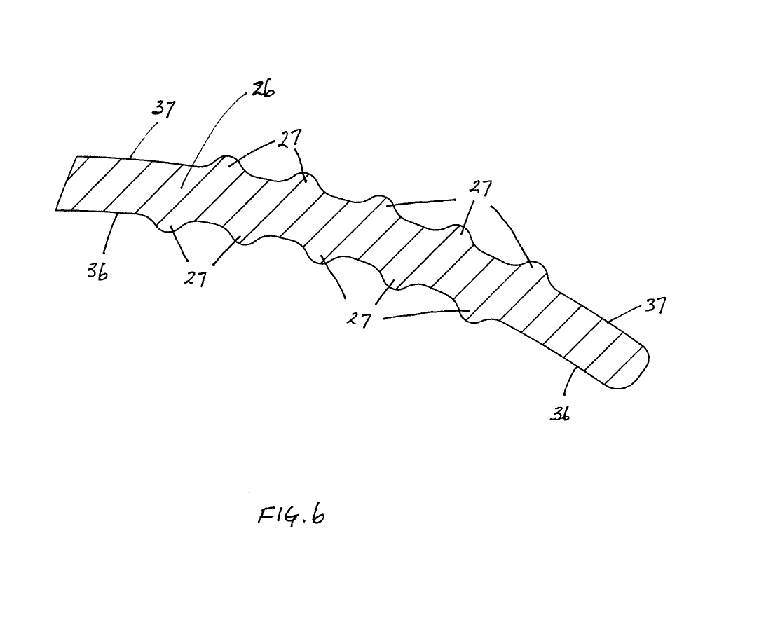

FIG. 6 shows an expanded view of detail D from FIG. 2, showing side view of the flap 26 from one embodiment of closure 10. More clearly visible in this view of the flap 26 are upper surface 36, lower surface 37, and optional ridges 27 of flap 26, which in this example shows ten ridges 27 that alternate on opposite surfaces of flap 26, five ridges 27 each on the upper surface 36 and lower surface 37. However, it will be understood that such ridges 27 can vary in number, height, and position along flap 26.

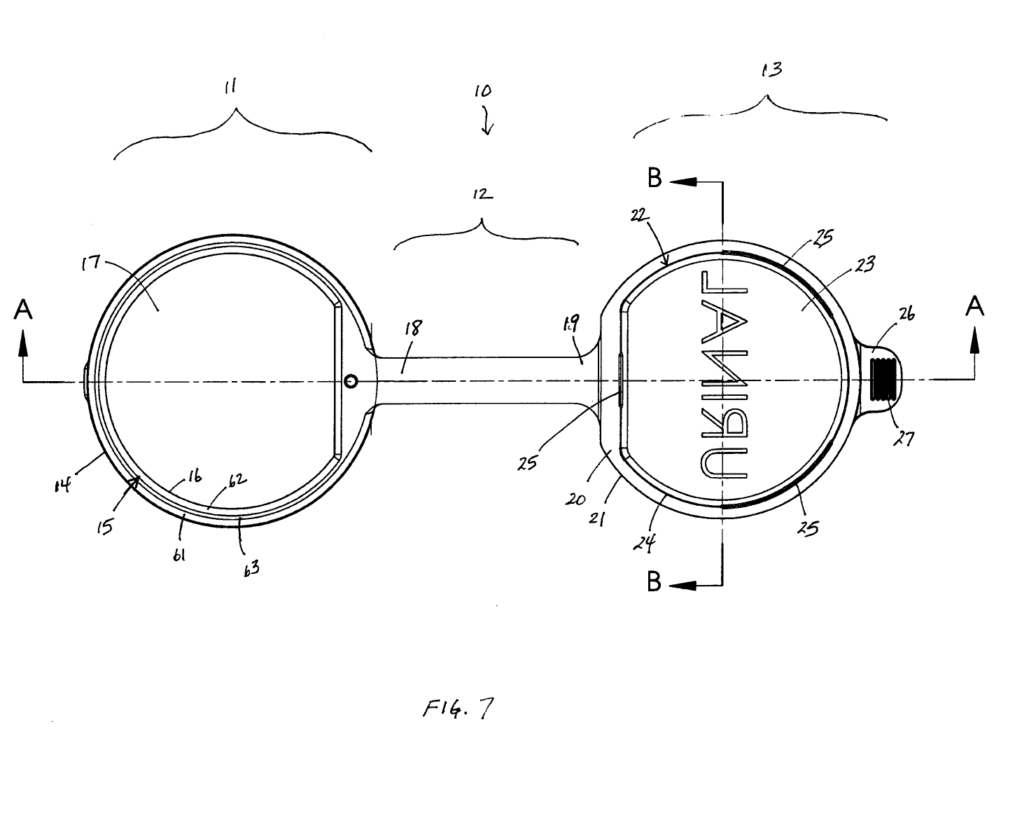

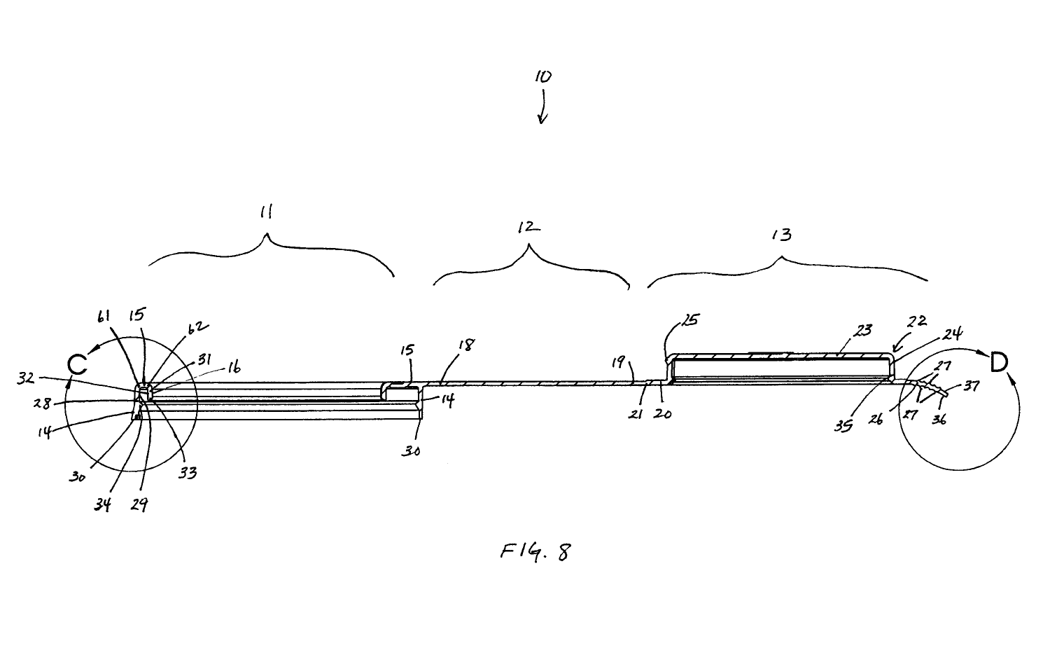

FIG. 7 shows a top view of another embodiment of the closure 10. Once again, the closure 10 generally comprises three sections, the ring 11, the hinge 12, and the lid 13. As used in FIG. 7, the “top view” of the closure 10 is a view from the top of the ring 11 and the bottom of the lid 13. Ring 11 comprises a side wall (not visible in this view) comprising an outer surface 14, an inner surface (not visible in this view), and bottom surface (not visible in this view), a top wall 15 comprising a first end 61, a second end 62, an upper surface 63, and a lower surface (not visible in this view), and lip (not visible in this view) having an outer surface 16, an inner surface (not visible in this view), and a tip (not visible in this view). The outer surface of the lip 16 defines an opening 17. Lid 13 comprises a base 20 having an outer edge 21, a raised central portion 22 comprising an upper end 23 having an outer wall 24 having optional protuberances 25, and a flap 26 having a series of optional ridges 27. The flap 26 is connected to the outer edge 21 of the base 20 of the lid 13. Opening 17 of ring 11 is adapted to receive the raised central portion 22 of lid 13. Although in FIG. 7 the opening 17 is shown as a circle flattened on one end, the opening (and corresponding raised central portion of lid) can take any shape, including, but not limited to, circular, oval, oval flattened on one end, triangular, rectangular, square, pentagonal, hexagonal, heptagonal, octagonal, polygonal, or any other desired shape. The hinge portion 12 comprises a first end 18 connected to the outer surface of the side wall 14 and a second end 19 connected to the outer edge 21 of the base 20 of the lid 13. In this embodiment of the closure 10, the first end of the hinge 18 is connected to the outer surface of the side wall 14 near the top wall 15. The connection of the hinge 12 to the ring 11 and the lid 13 is more clearly shown in FIG. 8. In this embodiment of the closure 10, the second end 19 of the hinge 12 is connected to the outer edge 21 of the base 20 of the lid 13 opposite (180° opposed to) the flap 26. However, in other embodiments of the closure 10 the second end 19 of the hinge 12 can be connected to the outer edge 21 of the base 20 of the lid 13 at a variety of different positions relative to the flap 26, including, but not limited to, about 15° opposed to, about 20° opposed to, about 25° opposed to, about 30° opposed to, about 35° opposed to, about 40° opposed to, about 45° opposed to, about 50° opposed to, about 55° opposed to, about 60° opposed to, about 65° opposed to, about 70° opposed to, about 75° opposed to, about 80° opposed to, about 85° opposed to, about 90° opposed to, about 95° opposed to, about 100° opposed to, about 105° opposed to, about 110° opposed to, about 115° opposed to, about 120° opposed to, about 125° opposed to, about 130° opposed to, about 135° opposed to, about 140° opposed to, about 145° opposed to, about 150° opposed to, about 155° opposed to, about 160° opposed to, about 165° opposed to, about 170° opposed to, or about 175° opposed to, or so.

FIG. 8 shows a side view of the embodiment of the closure 10 shown in FIG. 7 along line A-A. Once again closure 10 generally comprises three sections, ring 11, hinge 12, and lid 13. In this view, the side wall 28 of the ring 11 is shown, along with the outer surface 14, the inner surface 29 having an optional projection 34, and the bottom surface 30 of the side wall 28. Additionally, the top wall 15 comprising first end 61 and second end 62 is shown. Also shown is lip 31 having an outer surface 16, an inner surface 32 and a tip 33. The first end 18 of the hinge 12 is connected to the outer surface 14 of the side wall 28 near the top wall 15 of the ring 11, and the second end 19 of the hinge 12 is attached to the outer edge 21 of the base 20 of the lid 13. In this view, the raised portion 22 of the lid 13 is more clearly shown, with the upper end 23, a lower portion 35, and outer wall 24. Additionally, optional protuberance 25 on the outer wall 24 near the upper end 23 of the raised portion 22 of the lid 13 is shown. Also shown is flap 26 having an upper surface 36 and a lower surface 37, each of which include optional ridges 27. The number of the optional ridges 27 can vary from about 2 to about 10, including, but not limited to, 2, 3, 4, 5, 6, 7, 8, 9, 10, or more ridges. In other embodiments, the optional ridges 27 can be limited to only the upper surface 36 of the flap 26 or only the lower surface 37 of the flap 26. Additionally, although in this embodiments there are equal numbers of ridges 27 on the upper surface 36 and the lower surface 37 of the flap 26, in other embodiments there can be different numbers of ridges 27 on the upper surface 36 of and the lower surface 37 of the flap 26.

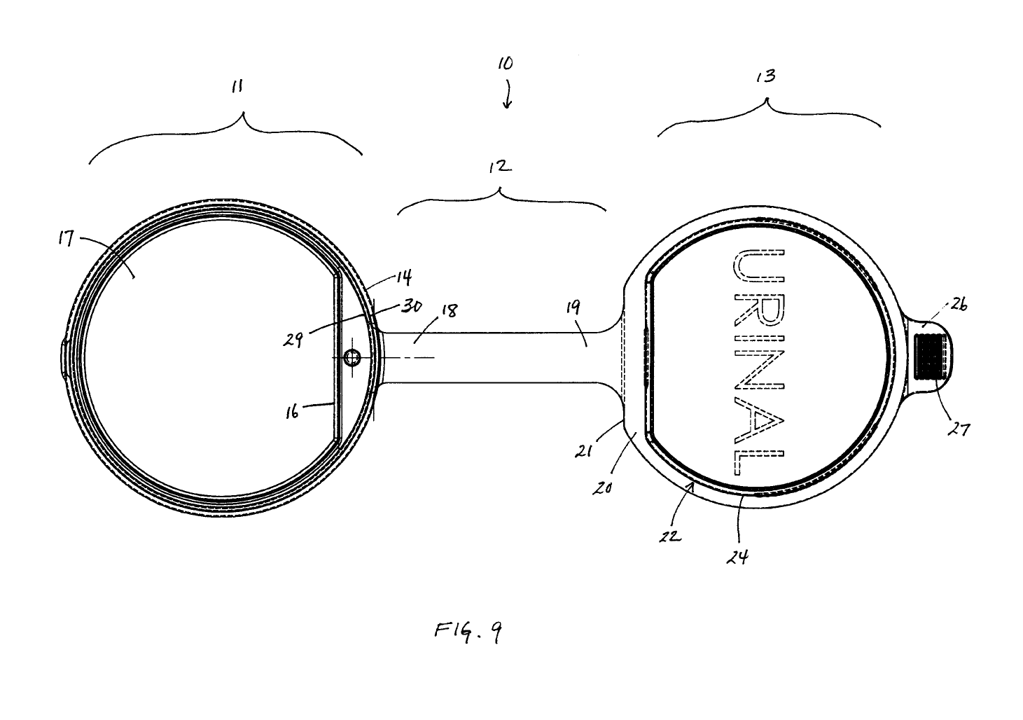

FIG. 9 shows a bottom view of the embodiment of the closure 10 shown in FIG. 7. Once again closure 10 generally comprises three sections, the ring 11, the hinge 12, and the lid 13. As used in FIG. 9, the “bottom view” of the closure 10 is a view from the bottom of the ring 11 and the top of the lid 13. Ring 11 comprises side wall (not visible in this view) comprising outer surface 14, inner surface 29, and bottom surface 30, top wall (not visible in this view), and lip (not visible in this view) having an outer surface 16 and a tip (not visible in this view). The outer surface 16 of the lip define an opening 17 that is adapted to receive a portion of the lid 13. The hinge portion 12 has a first end 18 connected to the outer surface 14 of the side wall near the top wall, and a second end 19 connected to the lid 13. The lid 13 has a base 20 having an outer edge 21 and a raised central portion 22 comprising an upper end (not visible in this view), a lower end (not visible in this view), an outer wall 24, and a flap 26 with a series of optional ridges 27. The second end 19 of the hinge portion 12 is connected to the outer edge 21 of the base 20 of the lid 13.

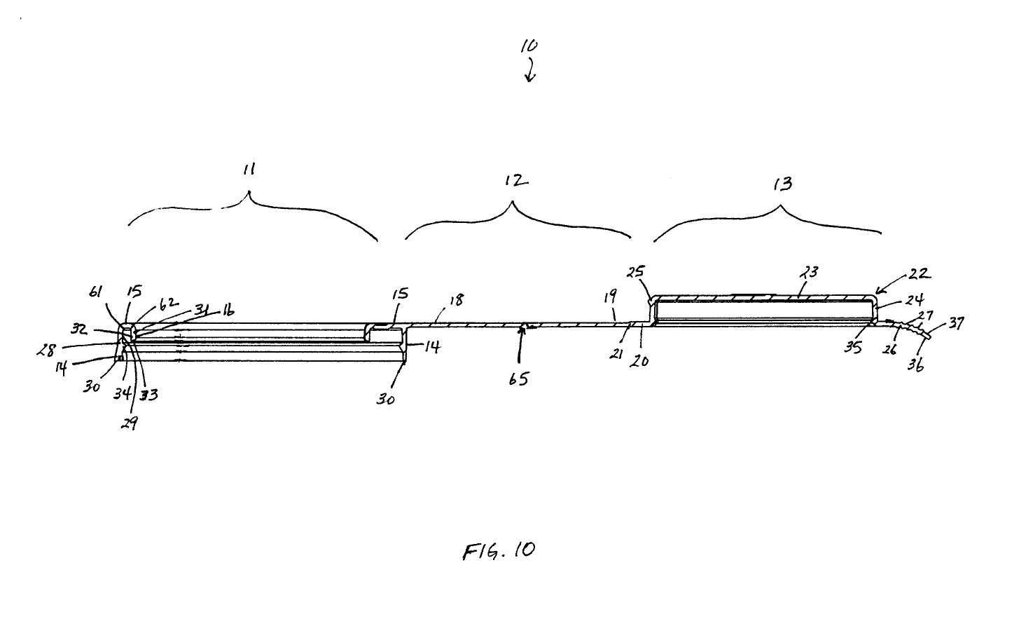

FIG. 10 shows a side view of another embodiment of the closure 10. Once again closure 10 generally comprises three sections, ring 11, hinge 12, and lid 13, although in this embodiment the hinge can be flexible or stiff In this view, the side wall 28 of the ring 11 is shown, along with the outer surface 14, the inner surface 29 having an optional projection 34, and the bottom surface 30 of the side wall 28. Additionally, the top wall 15 comprising first end 61 and second end 62 is shown. Also shown is lip 31 having an outer surface 16, an inner surface 32 and a tip 33. The first end 18 of the hinge 12 is connected to the outer surface 14 of the side wall 28 near the top wall 15 of the ring 11, and the second end 19 of the hinge 12 is attached to the outer edge 21 of the base 20 of the lid 13. Also included in this embodiment is indent 65 in hinge 12. Although indent 65 is shown in this embodiment with the first end 18 of the hinge 12 is connected to the outer surface 14 of the side wall 28 near the top wall 15 of the ring 11, it can also be used in embodiments wherein the first end 18 of the hinge 12 is connected to the outer surface 14 of the side wall 28 near the bottom surface 30 of the ring 11. In this view, the raised portion 22 of the lid 13 is more clearly shown, with the upper end 23, a lower portion 35, and outer wall 24. Additionally, optional protuberance 25 on the outer wall 24 near the upper end 23 of the raised portion 22 of the lid 13 is shown. Also shown is flap 26 having an upper surface 36 and a lower surface 37, each of which include optional ridges 27. The number of the optional ridges 27 can vary from about 2 to about 10, including, but not limited to, 2, 3, 4, 5, 6, 7, 8, 9, 10, or more ridges. In other embodiments, the optional ridges 27 can be limited to only the upper surface 36 of the flap 26 or only the lower surface 37 of the flap 26. Additionally, although in this embodiments there are equal numbers of ridges 27 on the upper surface 36 and the lower surface 37 of the flap 26, in other embodiments there can be different numbers of ridges 27 on the upper surface 36 of and the lower surface 37 of the flap 26.

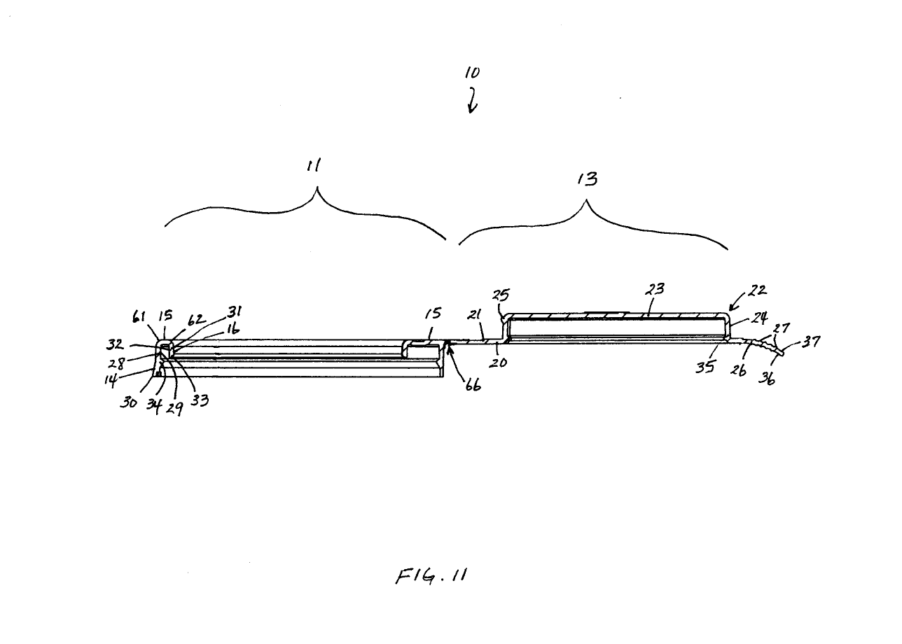

FIG. 11 shows a side view of another embodiment of the closure 10. In this embodiment, closure 10 generally comprises two sections, ring 11 and lid 13, connected by a generally pliable living hinge 66. In this view, the side wall 28 of the ring 11 is shown, along with the outer surface 14, the inner surface 29 having an optional projection 34, and the bottom surface 30 of the side wall 28. Additionally, the top wall 15 comprising first end 61 and second end 62 is shown. Also shown is lip 31 having an outer surface 16, an inner surface 32 and a tip 33. In this view, the raised portion 22 of the lid 13 is more clearly shown, with the upper end 23, a lower portion 35, and outer wall 24. Additionally, optional protuberance 25 on the outer wall 24 near the upper end 23 of the raised portion 22 of the lid 13 is shown. Also shown is flap 26 having an upper surface 36 and a lower surface 37, each of which include optional ridges 27. The number of the optional ridges 27 can vary from about 2 to about 10, including, but not limited to, 2, 3, 4, 5, 6, 7, 8, 9, 10, or more ridges. In other embodiments, the optional ridges 27 can be limited to only the upper surface 36 of the flap 26 or only the lower surface 37 of the flap 26. Additionally, although in this embodiments there are equal numbers of ridges 27 on the upper surface 36 and the lower surface 37 of the flap 26, in other embodiments there can be different numbers of ridges 27 on the upper surface 36 of and the lower surface 37 of the flap 26.

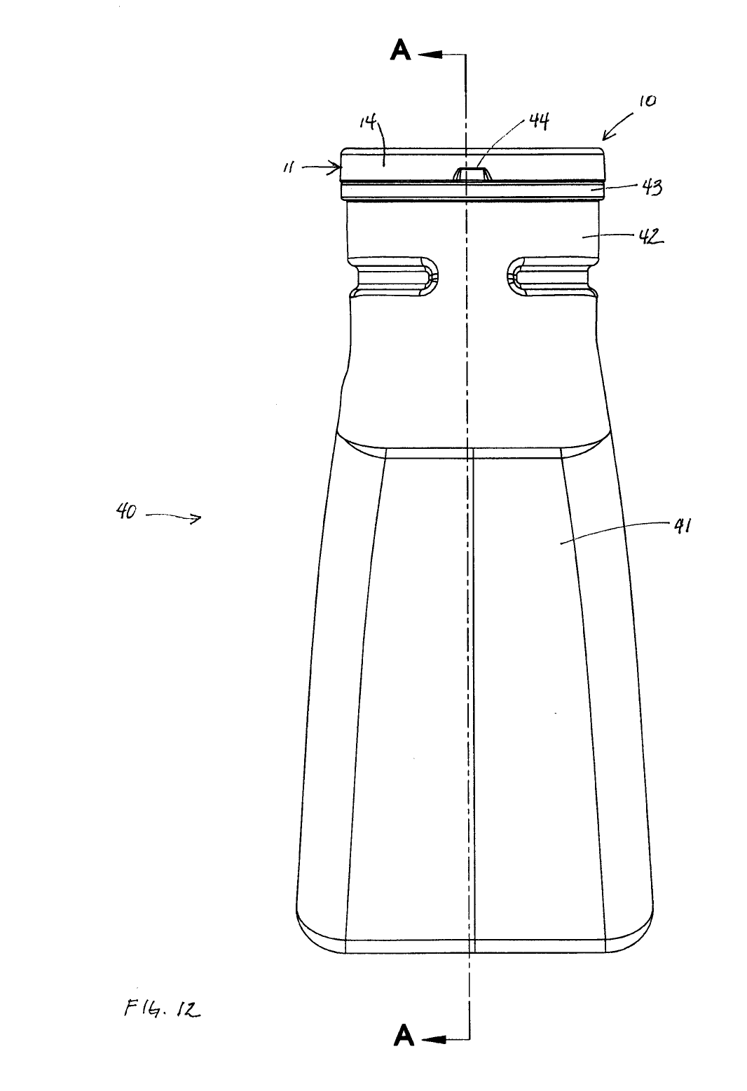

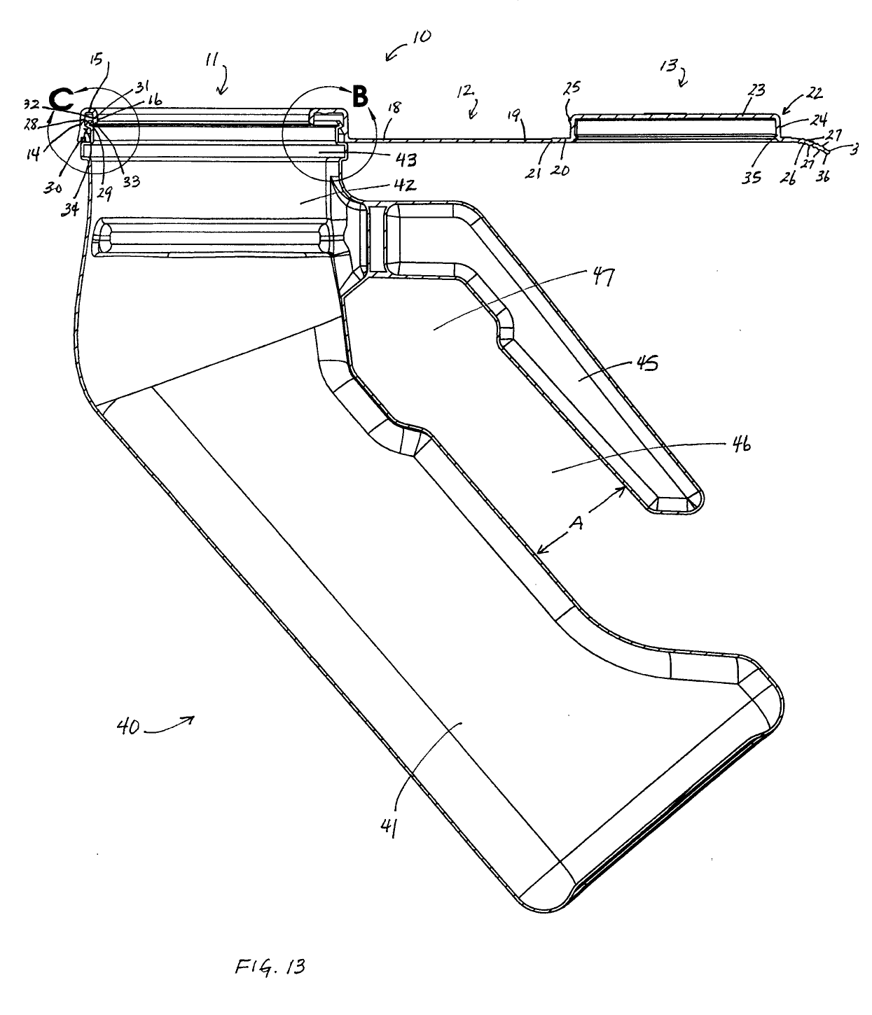

FIG. 12 shows a front view of one embodiment of a combination of closure 10 and blow-molded container 40, with the closure 10 in the open position. Blow-molded container 40 is shown as an integral, all plastic conventional male urinal having a body 41, neck 42 and rim 43. Shown in this view of closure 10 is ring 11 having outer surface 14 and optional bump-out 44. FIG. 13 shows a side view of one embodiment of the combination of closure 10 and blow-molded container 40, with the closure in the open position, through line A-A of FIG. 12. Container 40 comprises body 41, handle 45, neck 42, and rim 43. The handle 45 is generally parallel to the container 40 and defines an open area 46 and enlarged cove area 47 therebetween. The shape of the open area 46 generally has a narrow dimension “A” through which the narrow dimension of a bed rail (not shown) may pass as container 40 is fitted over the bed rail of a hospital bed (not shown). The dimensions of urinals and hospital bed rails are generally of a standard size such that the enlarged cove area 47 receives a wide dimension of the bed rail after it has passed through narrow dimension “A” of open area 46. Closure 10 comprises ring 11 comprising a side wall having outer surface 14, inner surface 29 having an optional projection 34, and bottom surface 30, top wall 15, and lip 31 having outer surface 16, inner surface 32 and tip 33, hinge 12 having a first end 18 and a second end 19, and lid 13 having a base 20 with outer edge 21, raised portion 22 having upper end 23, lower end 35, outer wall 24, and optional protuberance 25, and flap 26 having an upper surface 36 and a lower surface 37, each of which include optional ridges 27. In this embodiment, the first end 18 of the hinge 12 is connected to the outer surface 14 of the side wall 28 near the bottom surface 30 of the ring 11, and the second end 19 of the hinge 12 is attached to the outer edge 21 of the base 20 of the lid 13.

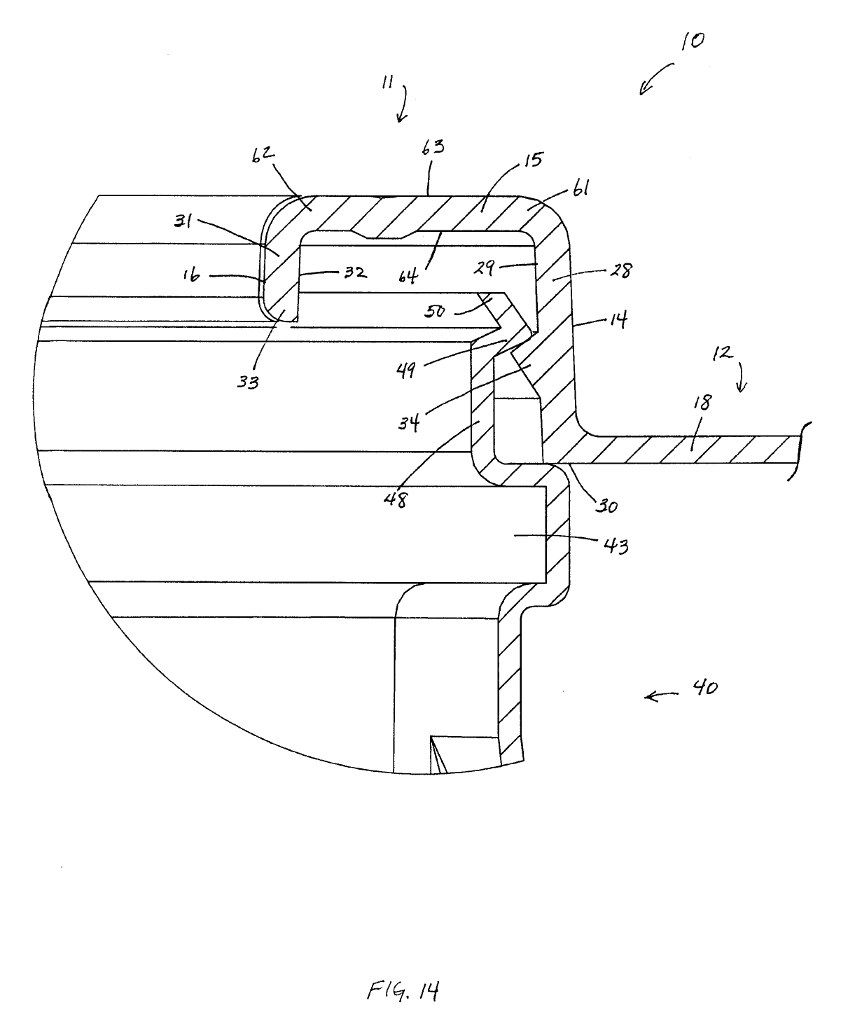

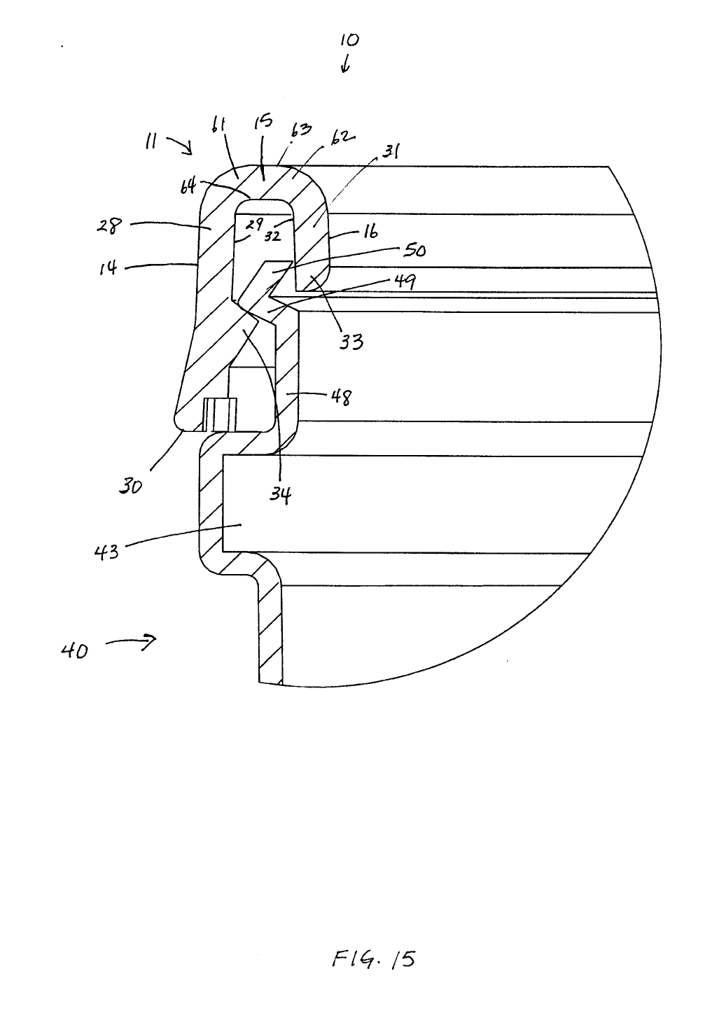

FIG. 14 shows an expanded view of detail B from FIG. 13, showing a side view of ring 11, hinge 12 having first end 18, and rim 43 of container 40. This view shows ring 11 comprising side wall 28 having outer surface 14, inner surface 29 having optional projection 34, and lower surface 30, top wall 15 having first end 61, second end 62, upper surface 63, and lower surface 64, and lip 31 with outer surface 16, inner surface 32, and tip 33. Additionally shown is connection of first end 18 of hinge 12 to outer surface 14 near the bottom surface 30 of side wall 28 of ring 11 of closure 10. Rim 43 of container 40 is shown having upward-projecting lip 48, attachment region 49, and untrimmed sharp projection 50. The ring 11 of closure 10 and rim 43 of container 40 are connected through engagement of projection 34 with attachment region 49, removing exposure to untrimmed sharp projection 50. FIG. 15 shows an expanded view of detail C from FIG. 13, showing side view of ring 11 and rim 43 of container 40. This view shows ring 11 comprising side wall 28 having outer surface 14, inner surface 29 having optional projection 34, and lower surface 30, top wall 15 having first end 61, second end 62, upper surface 63, and lower surface 64, and lip 31 with outer surface 16, inner surface 32, and tip 33. Rim 43 of container 40 is shown having upward-projecting lip 48, attachment region 49, and untrimmed sharp projection 50. In this embodiment, the ring 11 of closure 10 and rim 43 of container 40 are connected through engagement of projection 34 with attachment region 49, removing exposure to untrimmed sharp projection 50. However, in other embodiments the ring of the closure and the lip portion of the container are connected by ultrasonic welding, spin welding, through manual or mechanical securement, or through the use of an adhesive. Adhesives that can be used to connect the ring of the closure to the lip of the container include, but are not limited to, solvent-type binding glues or adhesives, acrylic cement, and hot melt moisture curing adhesives. In such embodiments, the projection 34 of the ring 11 and the attachment region 49 of the container 40 may be independently present or absent as desired.

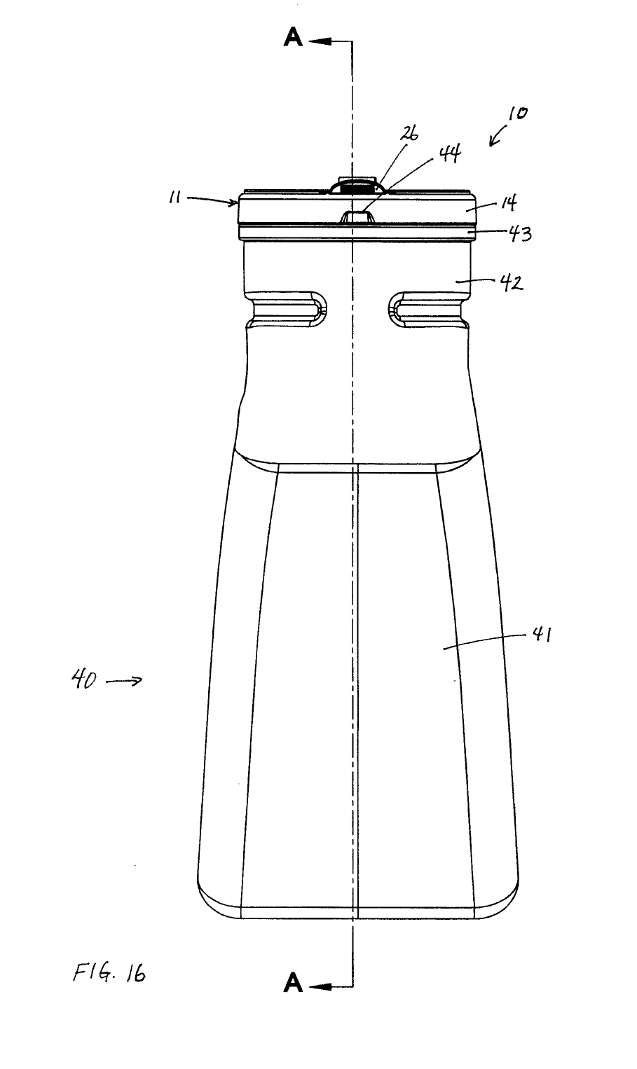

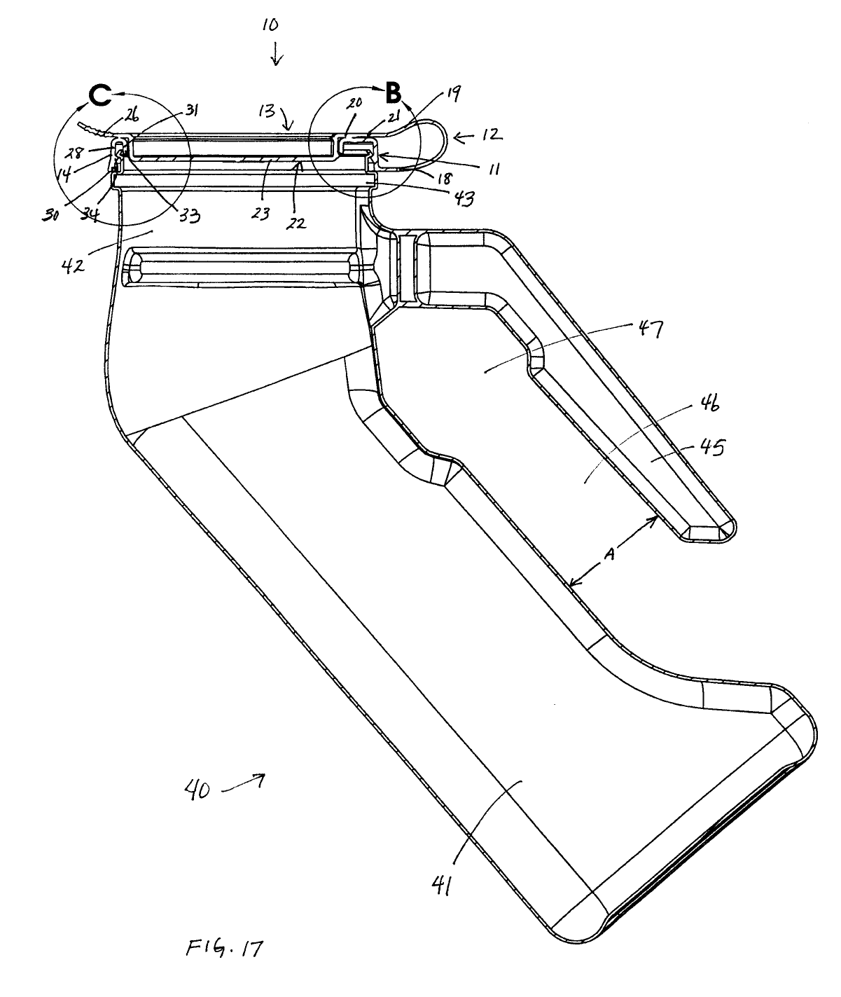

FIG. 16 shows a front view of one embodiment of a combination of closure 10 and blow-molded container 40, with the closure 10 in the shut or closed position. Once again blow-molded container 40 is shown as an integral, all plastic conventional male urinal having a body 41, neck 42 and rim 43. Shown in this view of closure 10 is ring 11 having outer surface 14, optional bump-out 44, and flap 26. FIG. 17 shows a side view of one embodiment of the combination of closure 10 and blow-molded container 40, with the closure in the shut or closed position, through line A-A of FIG. 16. Container 40 comprises body 41, handle 45, neck 42, and rim 43. The handle 45 is generally parallel to the container 40 and defines an open area 46 and enlarged cove area 47 therebetween. The shape of the open area 46 generally has a narrow dimension “A” through which the narrow dimension of a bed rail (not shown) may pass as container 40 is fitted over the bed rail of a hospital bed (not shown). The dimensions of urinals and hospital bed rails are generally of a standard size such that the enlarged cove area 47 receives a wide dimension of the bed rail after it has passed through narrow dimension “A” of open area 46. Closure 10 comprises ring 11 comprising side wall 28 having outer surface 14, optional projection 34, and bottom surface 30, lip 31 having tip 33, hinge 12 having a first end 18 and a second end 19, and lid 13 having a base 20 with outer edge 21, raised portion 22 having upper end 23, and flap 26. In this embodiment, the first end 18 of the hinge 12 is connected to the outer surface 14 of the side wall 28 near the bottom surface 30 of the ring 11, and the second end 19 of the hinge 12 is attached to the outer edge 21 of the base 20 of the lid 13.

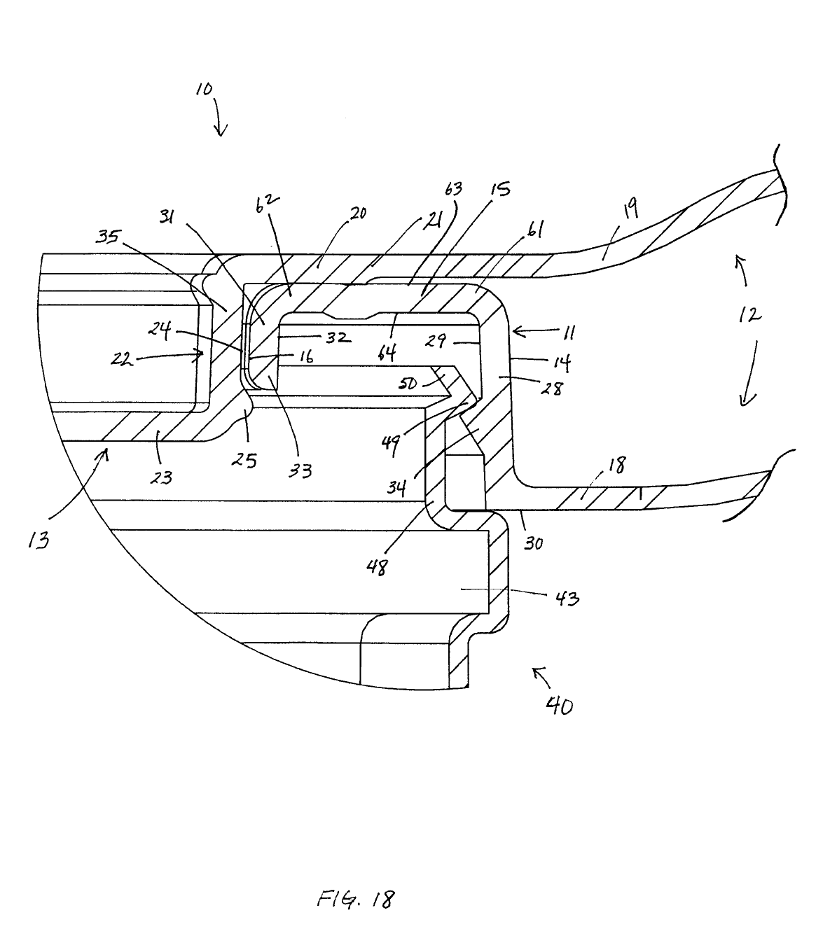

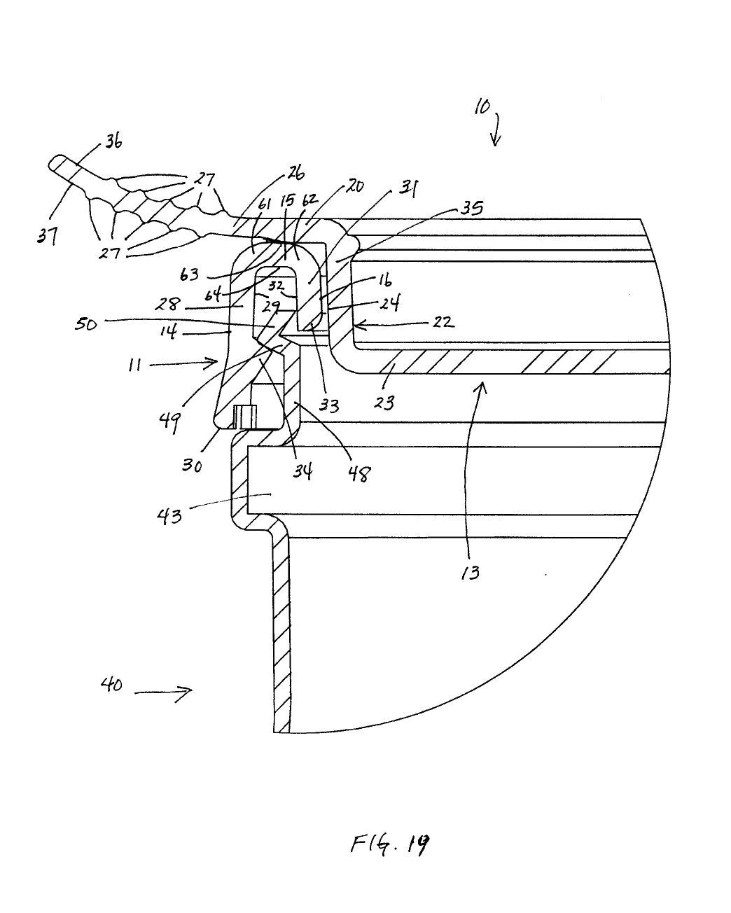

FIG. 18 shows an expanded view of detail B from FIG. 17, showing a side view of closure 10 having ring 11, hinge 12, lid 13, and rim 43 of container 40. This view shows ring 11 having side wall 28 comprising outer surface 14, inner surface 29 having optional projection 34, and lower surface 30, top wall 15 having first end 61, second end 62, upper surface 63, and lower surface 64, lip 31 having outer surface 16, inner surface 32, and tip 31, releasably-engaged lid 13 having base 20 with outer edge 21, raised portion 22 with upper end 23, lower end 35, and outer wall 24 with optional protuberance 25, and hinge 12 having first end 18 connected to outer surface 14 of side wall 28 near the lower surface 30 of side wall 28 and second end 19 connected to outer edge 21 of base 20 of lid 13. Rim 43 of container 40 is shown having upward-projecting lip 48, attachment region 49, and untrimmed sharp projection 50. The ring 11 of closure 10 and rim 43 of container 40 are connected through engagement of projection 34 with attachment region 49, removing exposure to untrimmed sharp projection 50. In addition, in the closed or shut position of closure 10 the outer surface 16 of lip 31 of ring 11 is releasably-engaged with the outer wall 24 of the raised portion 22 of lid 13. FIG. 19 shows an expanded view of detail C from FIG. 17, showing side view of ring 11, lid 13, and rim 43 of container 40. This view shows ring 11 having side wall 28 comprising outer surface 14, inner surface 29 having optional projection 34, and lower surface 30, top wall 15 having first end 61, second end 62, upper surface 63, and lower surface 64, lip 31 having outer surface 16, inner surface 32, and tip 31, and releasably-engaged lid 13 having base 20, raised portion 22 with upper end 23, lower end 35, and outer wall 24. Rim 43 of container 40 is shown having upward-projecting lip 48, attachment region 49, and untrimmed sharp projection 50. In this embodiment the ring 11 of closure 10 and rim 43 of container 40 are connected through engagement of projection 34 with attachment region 49, removing exposure to untrimmed sharp projection 50. In addition, in the closed or shut position of closure 10 the outer surface 16 of lip 31 of ring 11 is releasably-engaged with the outer wall 24 of the raised portion 22 of lid 13.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A closure, comprising:
a) a ring comprising:
i) a side wall comprising an outer surface, an inner surface, and a bottom surface;
ii) a top wall comprising a first end, a second end, an upper surface, and a lower surface, said side wall connected to said first end of said top wall; and
iii) a lip comprising an inner surface, an outer surface, and a tip, said lip connected to said second end of said top wall and defining an opening;
b) a hinge comprising a first end and a second end, said first end of said hinge connected to said outer surface of said side wall of said ring; and
c) a lid comprising:
i) a base comprising an outer edge; and
ii) a raised portion comprising an upper end, a lower end, and an outer wall, said lower end of said raised portion inset from said outer edge of said base and connected to said base, said raised portion adapted to releasably engage said lip of said ring, said outer edge of said base of said lid connected to said second end of said hinge.

2. The closure of claim 1, wherein said lip is generally parallel to said side wall.

3. The closure of claim 1, wherein said inner surface of said side wall comprises a projection.

4. The closure of claim 1, wherein said outer surface of said side wall of said ring further comprises a bump-out.

5. The closure of claim 1, wherein said hinge is flexible.

6. The closure of claim 1, wherein said hinge is a living hinge.

7. The closure of claim 1, wherein said hinge is stiff, and comprises an indent.

8. The closure of claim 1, wherein said lid further comprises a flap comprising an upper surface and a lower surface, said flap attached to said outer edge of said base of said lid.

9. The closure of claim 8, wherein said second edge of said flexible hinge and said flap attach to different regions of said outer edge of said base portion of said lid.

10. The closure of claim 8, wherein said flap comprises at least a first ridge.

11. The closure of claim 1, wherein said outer wall of said raised portion of said lid comprises at least a first protuberance.

12. The closure of claim 11, wherein said outer wall of said raised portion of said lid comprises a plurality of protuberances.

13. The closure of claim 1, wherein said closure is plastic.

14. A blow molded container comprising:
a) a body;
b) a rim connected to said body and defining an opening; and
c) a closure, said closure comprising:
i) a ring comprising:
(1) a side wall comprising an outer surface, an inner surface, and a bottom surface;
(2) a top wall comprising a first end, a second end, an upper surface, and a lower surface, said side wall connected to said first end of said top wall; and
(3) a lip comprising an inner surface, an outer surface, and a tip, said lip connected to said second end of said top wall and defining an opening;
said ring engaged with said rim of said container;
ii) a hinge comprising a first end and a second end, said first end of said hinge connected to said outer surface of said side wall of said ring; and
iii) a lid comprising:
(1) a base comprising an outer edge; and
(2) a raised portion comprising an upper end, a lower end, and an outer wall, said lower end of said raised portion inset from said outer edge of said base and connected to said base, said raised portion adapted to releasably engage said lip of said ring, said outer edge of said base of said lid connected to said second end of said hinge.

15. A blow molded container comprising:
a) a body;
b) a neck connected to said body;
c) a rim connected to said neck and defining an opening; and
d) a closure, said closure comprising:
i) a ring comprising:
(1) a side wall comprising an outer surface, an inner surface, and a bottom surface;
(2) a top wall comprising a first end, a second end, an upper surface, and a lower surface, said side wall connected to said first end of said top wall; and
(3) a lip comprising an inner surface, an outer surface, and a tip, said lip connected to said second end of said top wall and defining an opening;
said ring engaged with said rim of said container;
ii) a hinge comprising a first end and a second end, said first end of said hinge connected to said outer surface of said side wall of said ring; and
iii) a lid comprising:
(1) a base comprising an outer edge; and
(2) a raised portion comprising an upper end, a lower end, and an outer wall, said lower end of said raised portion inset from said outer edge of said base and connected to said base, said raised portion adapted to releasably engage said lip of said ring, said outer edge of said base of said lid connected to said second end of said hinge.

16. The container of claim 15, wherein said ring is releasably engaged with said container.

17. The container of claim 16, wherein said inner surface of said side wall of said ring further comprises a projection, and said projection engages said rim of said container.

18. The container of claim 15, wherein said ring is attached to said rim using ultrasonic welding, spin welding, manual or mechanical securement, or an adhesive.

19. The blow molded container of claim 15, wherein said blow molded container is a male urinal.

20. The blow molded container of claim 15, wherein said blow molded container is a female urinal.
